SEQUENCE LISTING (2 sequences)

SEQ ID NO: 1 - protein, 10 aa, Artificial Sequence
  Synthetic sequence
HAIMGVAFTW

SEQ ID NO: 2 - protein, 22 aa, Artificial Sequence
  Synthetic Sequence
HAIMGVAFTWVMALACAAPPLV

(19) United States
(12) Patent Application Publication
(10) Pub. No.: US 2017/0042985 A1
(43) Pub. Date: Feb. 16, 2017

(54) METHODS OF USING CYCLOOXYGENASE-PROSTACYCLIN SYNTHASE FUSION GENE

(71) Applicant: TEXAS HEART INSTITUTE, Houston, TX (US)

(72) Inventors: Richard A.F. DIXON, Houston, TX (US); Lei ZHOU, Houston, TX (US); Peter VANDERSLICE, Houston, TX (US); Ke-He RUAN, Bellaire, TX (US); Zhiqiang CHEN, Houston, TX (US)

(21) Appl. No.: 15/305,719

(22) PCT Filed: Apr. 22, 2015
(86) PCT No.: PCT/US15/27113

§ 371 Date: Oct. 21, 2016

Related U.S. Application Data
(60) Provisional application No. 61/983,335, filed on Apr. 23, 2014.

(51) Int. Cl. A61K 38/54; A61K 38/44; A61K 48/00; A61K 38/52

(52) U.S. Cl. CPC A61K 38/54; C12Y 114/99001; A61K 38/52; A61K 38/44; C12Y 503/99004; A61K 48/0075; A61K 48/0058

(57) ABSTRACT

An effective amount of a composition comprising (i) a plasmid having a cyclooxygenase-prostacyclin synthase fusion gene, and (ii) a carrier fluid for use in treating an individual having a vascular disease or at risk of developing a vascular disease. A composition comprising a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof, for use in treating an individual having a vascular disease or at risk of developing a vascular disease.

### CROSS-REFERENCE TO RELATED APPLICATION

The present application is the U.S. National Stage under 35 U.S.C. §371 of International Patent Application No. PCT/U52015/027113, filed Apr. 22, 2015, which claims priority to U.S. Provisional Patent Application No. 61/983,335, filed Apr. 23, 2014, the disclosures of which are hereby incorporated herein by reference.

### STATEMENT REGARDING SPONSORED RESEARCH

The invention described and claimed herein was made in part utilizing funds supplied by NIH NIHLB, Grant# 1RC1HL100807-1. The Government has certain rights in this invention.

### TECHNICAL FIELD

This disclosure relates to methods of treatment for vascular diseases. More specifically, it relates to compositions and methods of using a cyclooxygenase-prostacyclin synthase fusion gene for the treatment of vascular diseases.

### BACKGROUND

Vascular diseases are at the top of the list of most serious health problems, and are the leading cause of death in US. While the statistics might vary globally, vascular diseases are a cause of great worry around the world. Pulmonary arterial hypertension (PAH) and peripheral arterial disease (PAD) represent a couple of very concerning examples of vascular diseases.

PAH is a progressive disorder characterized by abnormally high blood pressure in the pulmonary artery, right ventricular (RV) overload, and, eventually, right heart failure leading to death. The estimated median survival of patients diagnosed with idiopathic PAH is <3 years. The primary treatment option that has been shown to increase the survival rate is the continuous systemic administration of prostacyclin (PGI2 or PGI₂); the median survival rate increases from 2.8 years in untreated patients to 5 years in treated patients. An oral formulation of treprostinil was recently approved for pulmonary arterial hypertension (PAH) by the U.S. Food and Drug Administration (FDA), but its efficacy is minimal and must be used in combination with other agents and it has not been tested for PAD.

However, systemic prostacyclin therapy has serious limitations, including a short halflife and the need for a permanent intravenous catheter. Other treatment options are needed. One alternative being studied is cell-based gene therapy using bone marrow-derived endothelial-like progenitor cells (ELPCs) that express endothelial nitric oxide synthase (eNOS). However, unlike synthetic prostacyclin and its analogs, eNOS treatment is not an established therapeutic approach and has not been included in the PAH treatment algorithm used by clinicians.

The growing prevalence of PAD is an increasing global concern as the population ages. PAD is an atherosclerotic disease associated with diabetes, hypertension, hypercholesterolemia, and coronary artery disease. Currently, PAD affects 12-14% of the general population, and its incidence is accelerating because of the increase in the elderly population. More than 10 million people in the United States have PAD. The 2 major clinical stages of PAD—intermittent claudication and critical limb ischemia (CLI)—result from insufficient blood supply to lower extremities, but the clinical outcome is more severe in the latter stage. Conventional treatments for PAD, such as angioplasty, stent deployment, and peripheral bypass surgery, are less effective when PAD progresses and causes obstruction of arterioles. In these cases, patients may develop untreatable claudication, rest pain, and ulcers that can progress to gangrene and other infections requiring amputation of a lower limb. Although surgical advancements have improved the lives of some PAD patients, many are not treated surgically because of the risk of complications. New therapeutic approaches are needed to promote vascular growth, reduce functional impairment of ischemic legs, and improve quality of life.

Exogenous prostacyclin replacement therapy offers a therapeutic alternative for patients who are poor candidates for surgical revascularization, such as high-risk patients (e.g., the elderly). Clinical studies have shown that PGI2 therapy is efficacious, but because PGI2 is an unstable compound with a circulating half-life of 1-2 minutes, this approach requires continuous intravenous or intraarterial infusion, which is associated with side effects and several potential complications. While continuous intravenous PGI2 therapy is effective, this approach is inconvenient for PAD patients, as PGI2 must be administered by using a continuous pump with an indwelling catheter. This delivery system is cumbersome and greatly reduces the patient's quality of life. Moreover, significant adverse events are associated with this delivery system; infection at the infusion site can lead to life-threatening complications. In addition, continuous infusion of PGI2 is a financial burden. Although stable PGI2 analogues have been developed and used clinically, they still require continuous intravenous or subcutaneous infusion.

Some studies show that a localized delivery approach in which a micro-osmotic pump is used to directly administer PGI2 to ischemic hindlimbs of mice may overcome the disadvantages of systemic PGI2 therapy. Local PGI2 delivery alleviates hindlimb ischemia by improving perfusion and promoting arteriolar growth. However, there are side effects and potential complications associated with this therapeutic method as well.

A new approach to effectively deliver PGI2 is urgently needed for treating patients with vascular diseases, such as for example PAH and/or PAD. As such, there exists a need for improved compositions of PGI2 delivery and methods of using same.

### BRIEF DESCRIPTION OF THE DRAWINGS

For a more complete understanding of the present disclosure and advantages thereof, reference will now be made to the accompanying drawings/figures in which:

FIG. 1 illustrates a schematic of biosynthesis of prostanoids (e.g., prostaglandins, such as prostaglandin D₂ (PGD₂), E₂ (PGE₂), F₂ (PGF₂), and I₂ (PGI₂) (prostacyclin), or thromboxane A₂ (TXA₂)) through coupling reactions of upstream cyclooxygenases (COXs) and downstream individual synthases;

FIG. 2A displays a graph of right ventricular systolic pressure (RVSP) over time in rats injected with monocrotaline (MCT);

FIG. 2B displays a schematic of an MCT induced rat pulmonary arterial hypertension (PAH) model;

FIG. 3A displays a graph of RVSP in various treatment groups of a MCT induced rat PAH model;

FIG. 3B displays a graph of right ventricle (RV) hypertrophy in various treatment groups of a MCT induced rat PAH model;

FIG. 4 displays images of brachial muscles of various treatment groups of a MCT induced rat PAH model;

FIG. 5 displays microscope images of a cross section of a MCT-rat brachial skeletal muscle, two weeks after treatment with plasmid, wherein the plasmid contains a fusion gene;

FIG. 6 displays microscope images of a longitudinal section of a MCT-rat brachial skeletal muscle, two weeks after treatment with plasmid, wherein the plasmid contains a fusion gene;

FIG. 7 displays microscope images of a section of a MCT-rat brachial skeletal muscle, two weeks after treatment with vehicle plasmid;

FIG. 8 displays microscope images of a section of a MCT-rat brachial skeletal muscle, two weeks after treatment with plasmid, isotype IgG control, wherein the plasmid contains a fusion gene; and

FIG. 9 displays microscope images of a section of a MCT-rat brachial skeletal muscle, three weeks after treatment with plasmid, wherein the plasmid contains a fusion gene.

### SUMMARY

Disclosed herein is an effective amount of a composition comprising (i) a plasmid having a cyclooxygenase-prostacyclin synthase fusion gene, and (ii) a carrier fluid for use in treating an individual having a vascular disease or at risk of developing a vascular disease.

Also disclosed herein is a composition comprising a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof, for use in treating an individual having a vascular disease or at risk of developing a vascular disease.

Further disclosed herein is a composition comprising a plasmid comprising a cyclooxygenase-prostacyclin synthase fusion gene, wherein the plasmid comprises a human muscle specific promoter, and phosphate buffer saline, for use in treating an individual having pulmonary arterial hypertension or at risk of developing pulmonary arterial hypertension, wherein the plasmid is present within the composition in an amount of from about 0.4 mg/ml to about 500 mg/ml; and wherein the composition is administered via an intramuscular injection into a brachial skeletal muscle.

Further disclosed herein is a composition for prostacyclin (PGI2) delivery, wherein the composition for PGI2 delivery comprises: a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques below, including the exemplary designs and implementations illustrated and described herein, but may be modified within the scope of the appended claims along with their full scope of equivalents.

Disclosed herein are embodiments of compositions for prostacyclin (PGI2 or PGI₂) delivery and methods of using the same. In an embodiment, the composition for PGI2 delivery may be used for targeted delivery of PGI2 in specific body areas, such as for example ischemic areas, wherein PGI2 may promote a repair function (e.g., a tissue repair function).

In an embodiment, the composition for PGI2 delivery may comprise a cyclooxygenase-prostacyclin synthase fusion gene and a carrier fluid. In an embodiment, the composition for PGI2 delivery may comprise a DNA sequence comprising the cyclooxygenase-prostacyclin synthase fusion gene. In an embodiment, the composition for PGI2 delivery may comprise a plasmid comprising the cyclooxygenase-prostacyclin synthase fusion gene. In an embodiment, the composition for PGI2 delivery may be administered to an individual having a vascular disease or at risk of developing a vascular disease. Each of the components of the composition for PGI2 delivery as well as methods of using same will be described in more detail herein.

In an embodiment, the composition for PGI2 delivery may generally increase the levels of PGI2 in a specific body area by enabling the overexpression of PGI2 in said body area. In an embodiment, the composition for PGI2 delivery may comprise a biologically or pharmacologically active compound. For purposes of the disclosure herein, a biologically active compound can be defined as a compound that interacts in some fashion with any living cell, tissue, and/or organism. For example, the cyclooxygenase-prostacyclin synthase fusion gene is a biologically active compound.

PGI2, a member of the prostaglandin family, is synthesized from arachidonic acid (AA) in a multistep process involving the enzymes cyclooxygenase-1 (COX-1) or cyclooxygenase-2 (COX-2) and prostacyclin synthase (PGIS). As a vasodilatory drug, PGI2 has multiple favorable properties for treating vascular diseases, such as for example PAH and/or PAD. In addition to mediating vascular homeostasis, PGI2 inhibits thrombosis and platelet aggregation.

The function of PGI₂ is primarily mediated by the PGI₂ receptor (IP) on the cell surface. The role of PGI₂ as an endogenous anti-thrombotic and vasodilative agent was confirmed with the experimental data generated in IP receptor-knockout mice. The IP-deficient mice developed without vascular problems in normal situations. However, an increased thrombotic tendency was observed in the IP-deficient mice when endothelial damage was induced. These findings indicate that the anti-thrombotic system mediated by PGI₂ is activated in response to vascular injury to minimize the effects of vascular injury. It has been reported that defects in the IP receptor of platelets has pathogenetic significance for developing atherosclerosis at an early age. The evidence was derived from a 10 year-old human diagnosed with an occluded left popliteal artery who also had a defect of her IP receptor. This defect appears to be genetically determined and to contribute to the development of atherosclerosis.

Accumulating evidence indicates a critical role for PGI2 in controlling stem cell recruitment and survival and in promoting angiogenesis. Patients with critical limb ischemia (CLI) have reduced numbers of circulating progenitor cells; however, after 4 weeks of treatment with a PGI2 analogue, such patients show increased levels of progenitor cells and pain relief Human outgrown endothelial progenitor cells (EPCs) may produce PGI2 and endogenous secretion of PGI2 by EPCs may facilitate vascular regeneration. In contrast, inhibiting PGI2 production in EPCs may reduce their proliferation, survival, and angiogenic capacity in ischemic hindlimbs. PGI2 signaling promotes the migration and recruitment of EPCs to injured vessels. Impaired function of EPCs is associated with decreased endogenous PGI2 synthesis and signaling PGI2 may have the ability to enhance the natural abilities of stem cells. The cell-protective property of PGI2 in vivo may attenuate cell loss by stimulating their plasticity to adapt to unfavorable environments.

In an embodiment, increasing or enhancing PGI2 biosynthesis in cells may improve the outcome of patients with vascular disease. Generally, biosynthesis, also known as biogenesis or anabolism, is a multi-step, enzyme-catalyzed process, wherein substrates are converted into more complex products. In biosynthesis, simple compounds are modified, converted into other compounds, or joined together to form macromolecules.

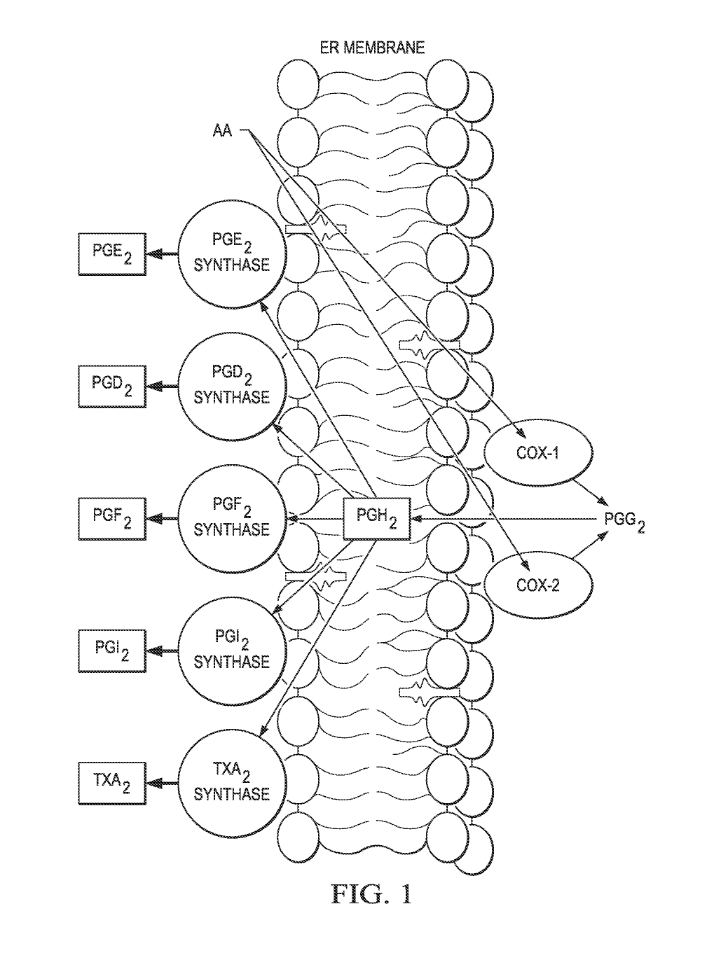

The recent discovery that COX-2 inhibitors may be linked to heart disease has greatly increased the interest in understanding the biology of COX enzymes, which convert a lipid molecule, AA, into different prostanoids (part of the eicosanoid family) having diverse and/or opposite biological functions. FIG. 1 shows a schematic of the biosynthesis of prostanoids. Biosynthesis of prostanoids generally comprises prostaglandins and thromboxane, formed via the COX pathway from AA in three catalytic (tri-catalytic) steps (represented by some of the thin line arrows in FIG. 1). AA may traverse across an endoplasmic reticulum (ER) membrane (e.g., from a first or cytoplasmic side of the ER membrane to a second or luminal side of the ER membrane) and be converted in catalytic step 1 to prostaglandin G₂ (PGG₂) by COX isoform-1 (COX-1) and/or COX-2, wherein COX-1 and COX-2 may be located on the luminal side of the ER membrane. In catalytic step 2, PGG₂ may be further converted to prostaglandin endoperoxide (prostaglandin H₂ (PGH₂)) by COX-1 and/or COX-2. PGH₂ may traverse across the ER membrane (e.g., from the luminal side of the ER membrane to the cytoplasmic side of the ER membrane). In catalytic step 3, PGH₂ may be further isomerized to biologically active end-products (prostaglandin D₂ (PGD₂), E₂ (PGE₂), F₂ (PGF₂), and I₂ (PGI₂ (prostacyclin)) or thromboxane A₂ (TXA₂) by individual synthases (PGD₂ synthase (PGDS), PGE₂ synthase (PGES), PGF₂ synthase (PGFS), and PGI₂ synthase (PGIS), or TXA₂ synthase (TXAS), respectively, as depicted in FIG. 1) in tissue specific manners, wherein such individual synthases may be located on the cytoplasmic side of the ER membrane. Prostanoids act as local hormones in the vicinity of their production site to regulate hemostasis and smooth muscle functions. Unlike the stable level of COX-1 expression, COX-2 expression is inducible and it responds to the stimuli of pro-inflammatory and other pathogenic factors. TXA₂ produced from PGH₂ by TXA₂ synthase (TXAS) has been implicated in various pathophysiological conditions as a proaggregatory and vasoconstricting mediator. PGI₂ is the main AA metabolite in vascular walls and has opposing biological properties to TXA₂, representing the most potent endogenous vascular protector acting as an inhibitor of platelet aggregation and a strong vasodilator on vascular beds. PGE₂ exhibits a variety of biological activities in inflammation. Aspirin and non-steroidal anti-inflammatory drugs (NSAID) inhibit both COX-1 and COX-2 activities to reduce the production of all prostanoids, which leads to thinning of the blood by reducing TXA₂ production and the suppression of inflammation through decreasing PGE₂ production. The selective COX-2 inhibiting drugs exhibit anti-inflammatory effects similar to aspirin and NSAIDs, but they may also promote strokes and heart attacks by decreasing the production of PGI₂, and increasing the production of TXA₂. This may occur because, when the COX-2 enzyme was specifically inactivated by COX-2 inhibitors, the PGH₂ produced by COX-1 was still available to be converted into other prostanoids such as TXA₂ by TXAS, leading to an increased risk of thrombosis and vasoconstriction.

Recently, PGI₂ has also been determined to be a ligand for the nuclear hormone receptor peroxisome proliferator-activated receptor (PPAR). Three PPAR-isoforms, PPARα, β/δ and γ have been cloned and implicated in the regulation of the expression of genes involved in lipid metabolism.

In both skeletal and cardiac muscle cells it has been demonstrated that the metabolic conversion of fatty acids is under control by PPARs. PGI₂ can effectively induce DNA binding and transcriptional activation by PPAR. PGI₂, acting as a ligand for PPARδ, induces increased expression of PPARδ in the arterial wall after a balloon injury, suggesting that PGI₂ effects vasodilation and anti-platelet aggregation through the IP receptor and PPARδ. It has also been speculated that PGI₂, as a ligand for PPARδ, induces anti-inflammatory activity in vascular diseases, such as atherosclerosis.

In an embodiment, peroxisome proliferator-activated receptor-beta/delta (PPARδ) can be a potential regulator of PGI2 signaling. In the search for endogenous targets for PGI2 signaling, PPARδ was found to colocalize with COX-2/PGIS and actively respond to PGI2 agonists. PPARδ is a ligand-activated nuclear hormone receptor that is ubiquitously expressed in various tissues. It forms heterodimers with retinoid X receptor, which binds to the peroxisome proliferator response element in the promoter region of target genes to control transcription. Emerging evidence suggests that PPARδ plays a critical role in stem cell survival and neovascularization. Accordingly, activation of PPARδ by PGI2 may promote stem cell-mediated vascular regeneration in ischemic hindlimbs. Inhibition of PPARδ by selective antagonists or specific siRNA in human progenitor cells may reduce PGI2-induced regenerative ability and blood vessel formation. PGI2, in partnership with PPARδ, accelerates embryo implantation and blastocyst hatching. In addition to its pro-survival and pro-angiogenic roles, PPARδ is important in adaptive responses to environmental changes. As a metabolic sensor, PPARδ regulates several metabolic genes involved in cellular homeostasis. PPARδ may play a critical role in mitochondrial function. In an embodiment, PGI2-PPARδ axis may affect the ability of stem cells to adjust to environmental changes.

In an embodiment, the carrier fluids that may be used in the composition for PGI2 delivery include any carrier fluid suitable for delivery of a biologically active compound in vivo. In an embodiment, the carrier fluid comprises a pharmaceutically acceptable carrier. For purposes of the disclosure herein, a “pharmaceutically acceptable carrier” is meant to encompass any carrier that does not interfere with effectiveness of a biological activity of any active ingredient (e.g., cyclooxygenase-prostacyclin synthase fusion gene) and that is not toxic to an individual to which it is administered. “Pharmaceutically acceptable” as used herein adheres to the U.S. Food and Drug Administration guidelines.

In an embodiment, the composition for PGI2 delivery may comprise an aqueous carrier fluid. In some embodiments, the aqueous carrier fluid may comprise deionized water. In other embodiments, the carrier fluid may comprise a saline solution (e.g., phosphate buffer saline). In yet other embodiments, the carrier fluid may comprise an aqueous buffer, such as for example phosphate buffer saline (PBS). In an embodiment, the carrier fluid may be included within the composition for PGI2 delivery in a suitable amount.

Nonlimiting examples of additives suitable for use in the carrier fluid in the present disclosure include solubilizing agents, stabilizing agents, preservatives such as for example anti-oxidants, and the like, or combinations thereof.

In an embodiment, the composition for PGI2 delivery may comprise a cyclooxygenase-prostacyclin synthase fusion gene. In such embodiment, the cyclooxygenase-prostacyclin synthase fusion gene may encode for a triple catalytic enzyme.

In an embodiment, the composition for PGI2 delivery may comprise a DNA sequence encoding for the triple catalytic enzyme, a cDNA sequence encoding for the triple catalytic enzyme, a plasmid comprising a DNA sequence encoding for the triple catalytic enzyme, a fusion gene encoding for the triple catalytic enzyme, a cyclooxygenase-prostacyclin synthase fusion gene, and the like, or combinations thereof

In an embodiment, PGI2 may be delivered by inducing PGI2 overexpression, e.g., expression of high levels of PGI2 or expression of PGI2 levels that are higher than regular PGI2 levels expressed in the absence of inducing PGI2 overexpression. A system that increases PGI2 biosynthesis in cells of the ischemic areas would help prevent the adverse events caused by conventional PGI2 delivery methods. As will be appreciated by one of skill in the art, and with help of this disclosure, effective and stable biosynthesis of PGI2 requires an increase in the expression of COX-1 or COX-2 in conjunction with PGIS, as illustrated in FIG. 1.

In an embodiment, PGI2 overexpression may be induced by composition for PGI2 delivery comprising a DNA sequence encoding for the triple catalytic enzyme. In an embodiment, the triple catalytic enzyme may enhance the expression of PGI2 in cells.

Recent studies of the structure and function relationship of COX enzymes and PGIS have advanced knowledge of the molecular mechanisms involved in the biosynthesis of PGI2 in native cells. Crystallographic studies of detergent-solubilized COX-1 and COX-2 suggest that the catalytic domains of the proteins lie on the luminal side of the endoplasmic reticulum (ER) and are anchored to the ER membrane by hydrophobic side chains of amphipathic helices A-D. These hydrophobic side chains of the putative membrane anchor domains also form an entrance to the substrate-binding channel and potentially form an initial docking site for the lipid substrate, AA. Recent progress in the topology and structural studies of human PGIS and TXAS have led to the proposal of models in which PGIS and TXAS have catalytic domains on the cytoplasmic side of the ER, opposite the orientation of COXs. In this configuration, the substrate channels of all the three enzymes, COX, PGIS and TXAS, open at or near the ER membrane surface. The coordination between COXs and PGIS or TXAS in the biosynthesis of TXA₂ and PGI2 may be facilitated by the enzyme's anchoring in the lipid membrane. The physical distances between COXs and PGIS are very small. Consequently, it should be possible to create a single protein molecule containing COX and PGIS sequences with minimum alteration of both enzymes' folding and membrane topologies by extending the N-terminal membrane anchor domain of PGIS using a transmembrane sequence linked to the COX-1 or COX-2, which then adopts the functions of both enzymes of COX and PGIS. In this case, AA could be directly converted into the vascular protector, PGI2, with concurrently decreasing the production of the unwanted PGE₂ and TXA₂.

In an embodiment, the triple catalytic enzyme may be characterized by a formula COX-linker-ES, wherein COX comprises a cyclooxygenase (COX) amino acid sequence, such as for example COX-1 or COX-2; wherein ES comprises an eicosanoid-synthesizing (ES) enzyme amino acid sequence; wherein the linker comprises from about 10 to about 22 amino acid residues of a transmembrane sequence; wherein the linker may be disposed between the COX and the ES; and wherein the linker may directly connect the COX to the ES. In an embodiment, the triple catalytic enzyme comprises a hybrid protein or hybrid peptide. For purposes of the disclosure herein, the term “about,” when used in conjunction with a percentage or other numerical amount, means plus or minus 10% of that percentage or other numerical amount. For example, the term “about 10 amino acid residues,” would encompass 10 amino acid residues plus or minus 1 amino acid residue.

In some embodiments, the linker (e.g., linker peptide) may function as a transmembrane linker in a cell, such that folding ability and function of each enzyme (e.g., COX, ES) of the triple catalytic enzyme may be substantially unaltered compared to the folding ability and function of respective native enzymes. As will be appreciated by one of skill in the art, and with the help of this disclosure, the linker is a peptide, since it comprises a relatively short sequence of amino acids. For purposes of the disclosure herein, the terms “linker” and “linker peptide” can be used interchangeably.

In an embodiment, the linker (e.g., linker sequence) comprises His-Ala-Ile-Met-Gly-Val-Ala-Phe-Thr-Trp (SEQ ID NO. 1) or His-Ala-Ile-Met-Gly-Val-Ala-Phe-Thr-Trp-Val-Met-Ala-Leu-Ala-Cys-Ala-Ala-Pro-Pro-Leu-Val (SEQ ID NO. 2). In certain embodiments, the linker sequence comprises residues 1-11, 1-12, 1-13, 1-14, 1-15, 1-16, 1-17, 1-18, 1-19, 1-20 or 1-21 of SEQ ID NO. 2. In some embodiments, the linker peptide provides approximately 10 A separation between the catalytic sites of the COX and the ES enzyme. In an embodiment, the connected enzymes (e.g., COX, ES) are preferably capable of substantially normal folding and enzymatic activity compared to the native folding and enzymatic activity of the native COX and ES enzymes.

In an embodiment, the triple catalytic enzyme may be characterized by a faster turnover rate when compared to a mixture of the native COX and ES enzymes. The hybrid protein (e.g., COX-linker-ES) does not only possess the individual enzymes' activities, but has a faster turnover rate as compared to a mixture of separate COX and ES enzymes.

In an embodiment, the ES may comprise PGIS or a downstream synthase thereof In an embodiment, the PGIS downstream synthase may comprise prostaglandin E synthase (PGES), prostaglandin D synthase (PGDS), or prostaglandin F synthase (PGFS). In an embodiment, the triple catalytic enzyme may be characterized by formulas COX-linker-PGIS, COX-linker-PGES, COX-linker-PGDS, or COX-linker-PGFS, wherein COX comprises COX-1 or COX-2. In such embodiment, the triple catalytic enzyme combines the enzymatic functions of COX (e.g., COX-1, COX-2) and ES (e.g., PGIS, PGES, PGDS, PGFS) in a single hybrid protein.

In an embodiment, the triple catalytic enzyme may be characterized by a formula COX-linker-PGIS, wherein COX comprises COX-1 or COX-2. In an embodiment, the COX-linker-PGIS may adopt the functions of COX and PGIS. In an embodiment, the COX-linker-PGIS may be able to continually convert AA into prostaglandin G₂ (catalytic step 1), prostaglandin H₂ (catalytic step 2) and prostacyclin (PGI2; catalytic step 3), wherein the catalytic steps have been described previously herein. Such conversion of AA into PGI2 may be even faster than coupling reactions using unlinked, co-expressed COX and PGIS.

In some embodiments, the triple catalytic enzyme may be characterized by formula COX-1linker-PGIS. In other embodiments, the triple catalytic enzyme may be characterized by formula COX-2linker-PGIS. In an embodiment, the triple catalytic enzyme may catalyze the three catalytic steps (e.g., three key reactions) in the biosynthesis of PGI2, thereby enhancing the expression of PGI2 (e.g., increasing the production of PGI2). In such embodiment, the triple catalytic enzyme links COX-1 or COX-2 to PGIS and catalyzes three key reactions for efficient production of PGI2 from AA.

In some embodiments, the COX-1-linker-PGIS protein may comprise an 130 kDa recombinant protein, wherein the recombinant protein may be constructed by linking together human cyclooxygenase (COX) isoform-1 (COX-1) and PGIS via a linker. In such embodiments, the linker may comprise from 10 to 22 amino acid residues of a transmembrane sequence, as previously described herein. In an embodiment, the COX-1-linker-PGIS protein may comprise COX-1-10aa-PGIS, wherein the linker comprises a 10 amino acid (10aa) transmembrane sequence (e.g., SEQ ID NO. 1). As will be appreciated by one of skill in the art, and with the help of this disclosure, some COX-2 inhibitors inhibit COX-2 activity but not COX-1 activity. Thus, the introduction of the COX-1-linker-PGIS hybrid protein to vascular systems is expected to help overcome the damage of the vascular functions caused by COX-2 inhibitors. In an embodiment, the triple catalytic enzyme may be characterized by formula COX-1-10aa-PGIS.

In other embodiments, the COX-2-linker-PGIS protein may comprise an 130 kDa recombinant protein, wherein the recombinant protein may be constructed by linking together human cyclooxygenase (COX) isoform-2 (COX-2) and PGIS via a linker. In such embodiments, the linker may comprise from 10 to 22 amino acid residues of a transmembrane sequence, as previously described herein. In an embodiment, the COX-2-linker-PGIS protein may comprise COX-2-10aa-PGIS, wherein the linker comprises a 10 amino acid (10aa) transmembrane sequence (e.g., SEQ ID NO. 1). In an embodiment, the triple catalytic enzyme may be characterized by formula COX-2-10aa-PGIS. The triple catalytic enzyme and methods of producing and/or using same are described in more detail in U.S. Publication No. 20100015120 A1, which is incorporated by reference herein in its entirety.

In an embodiment, the composition for PGI2 delivery comprising the cyclooxygenase-prostacyclin synthase fusion gene may be delivered locally in vivo by any suitable method, such as for example by injection (e.g., intramuscular injection). In an embodiment, once the composition for PGI2 delivery enters an organism, such composition may contact cells of said organism, and the cyclooxygenase-prostacyclin synthase fusion gene may enter (e.g., be transfected into, be incorporated into, etc.) the cells of said organism, to yield transfected cells. In such embodiment, the transfected cells may overexpress (e.g., express high levels of) PGI2.

In an embodiment, the cyclooxygenase-prostacyclin synthase fusion gene may be introduced in cells via any suitable transfection methods, such as for example by using a plasmid comprising a DNA sequence encoding for the triple catalytic enzyme. Generally, plasmids are small circular, double-stranded DNA molecules. Plasmids may be used for insertion of therapeutic genes (e.g., a fusion gene) at pre-selected chromosomal target sites within the human genome. Plasmids are capable of entering a host cell and automatically replicating within the host cell. In such embodiment, the plasmid comprising the cyclooxygenase-prostacyclin synthase fusion gene may be used for COX gene therapy.

In an embodiment, the plasmid may comprise a DNA sequence encoding for a COX, a transmembrane linker peptide, and an ES. In some embodiments, COX comprises COX-1. In other embodiments, COX comprises COX-2. In an embodiment, ES comprises PGIS. In an embodiment, the linker directly connects the COX to the ES. In an embodiment, the plasmid comprises a DNA sequence encoding for the triple catalytic enzyme, and such DNA sequence may be referred to as a “fusion gene.”

In an embodiment, the plasmid may comprise a promoter. In an embodiment, the plasmid may comprise a constitutively active mammalian promoter. Generally, a constitutive promoter is active in all circumstances in the cells, as opposed to regulated promoters that become active in response to a specific stimulus. As will be appreciated by one of skill in the art, and with the help of this disclosure, constitutively active means the promoter constantly drives the transgene expression.

Nonlimiting examples of promoters suitable for use in the present disclosure include a human muscle specific promoter, human muscle creatinine kinase promoter, human □-skeletal actin promoter, human desmin promoter, human troponin I promoter, human cytomegalovirus promoter, and the like.

In an embodiment, the plasmid may comprise an inducible promoter. Generally, the activity of inducible promoters can be induced by the presence or absence of exogenous (i.e., external) factors. As will be appreciated by one of skill in the art, and with the help of this disclosure, the expression of genes operably linked to inducible promoters can be turned on or off at certain stages of development of an organism or in a particular tissue. In an embodiment, inducible promoters can respond (e.g., activate) to chemical compounds, such as for example antibiotics (e.g., doxycycline).

Nonlimiting examples of inducible promoters suitable for use in the present disclosure include antibiotic-inducible promoter, tetracycline-inducible promoter, doxycycline-inducible promoter, bacitracin-inducible promoter, vancomycin-inducible promoter, isopropyl beta-D-thiogalactopyranoside (IPTG)-inducible promoter, and the like. In an embodiment, an inducible promoter can comprise one or more components of RHEOSWITCH THERAPEUTIC SYSTEM® (RTS®), wherein RTS® is a platform that can be applied to create high value therapeutics through controlled cellular expression of therapeutic proteins of interest, and is available from Intrexon.

In an embodiment, the plasmid may be included within the composition for PGI2 delivery in a suitable amount (e.g., an effective amount). In an embodiment, the plasmid may be present within the composition for PGI2 delivery in an amount of from about 0.4 mg/ml to about 500 mg/ml, alternatively from about 0.5 mg/ml to about 250 mg/ml, or alternatively from about 1 mg/ml to about 100 mg/ml, based on the total volume of the composition for PGI2 delivery. In an embodiment, the plasmid may be present within the composition for PGI2 delivery in an amount of about 2 mg/ml, based on the total volume of the composition for PGI2 delivery.

In an embodiment, the composition for PGI2 delivery excludes a viral vector. Generally, viral vectors are virus-based tools used for delivering genetic material (e.g., a gene) into a cell. Nonlimiting examples of viral vectors include viral expression vectors, such as for example a retrovirus, a lentivirus, an adenovirus, an adeno-associated virus, etc.

In some embodiments, the triple catalytic enzyme may be produced by a host cell containing an expressible DNA sequence encoding for the triple catalytic enzyme, wherein such expressible DNA sequence was introduced into the host cell by the plasmid. In an embodiment, the host cell may be transfected with a vector comprising the DNA sequence encoding for the triple catalytic enzyme to produce host cell containing an expressible DNA sequence encoding for the triple catalytic enzyme. In some embodiments, the host cell comprises a cell within an individual treated with the composition for PGI2 delivery, and such host cell may be located within and/or proximate a body area where the composition for PGI2 delivery was administered to the individual. In such embodiments, the host cell may produce the triple catalytic enzyme. In an embodiment, the triple catalytic enzyme comprises enzymatically active cyclooxygenase, transmembrane linker, and enzymatically active prostacyclin synthase.

In an embodiment, the composition for PGI2 delivery may be prepared via any suitable method or process. The components of the composition for PGI2 delivery (e.g., plasmid, carrier fluid) may be combined using any mixing device compatible with the composition, e.g., that does not alter or destroy the composition for PGI2 delivery components, such as the plasmid.

In some embodiments, the composition for PGI2 delivery may be prepared by using plasmid carriers to enhance in vivo gene transfection efficiency. Nonlimiting examples of plasmid carriers suitable for use in the present disclosure include a liposome carrier, a nanoparticle carrier, polyethyleneimine (PEI), and the like, or combinations thereof.

In an embodiment, the composition for PGI2 delivery may be used for the treatment of an individual having a vascular disease or at risk of developing a vascular disease, wherein the composition for PGI2 delivery may be a pharmaceutical composition. In an embodiment, the vascular disease may comprise PAH, PAD, peripheral vascular disease, chronic obstructive pulmonary disease (COPD), ischemia, limb ischemia, CLI, Reynaud's syndrome, ischemic stroke, myocardial infarction, systemic hypertension, stroke, subarachnoid hemorrhage, or combinations thereof.

In an embodiment, a method of treating an individual having a vascular disease or at risk of developing a vascular disease may comprise administering to the individual an effective amount of the composition for PGI2 delivery, alone or in combination with other therapies, wherein the composition for PGI2 delivery may be a pharmaceutical composition, thereby ameliorating, deterring and/or preventing the vascular disease in said individual. Where other therapies are envisioned, they may include, without limitation, combination with other PGI2 stable precursors or analogues (e.g., Cicaprost, Iloprost, Beraprost, Carbaprostacyclin, Trepostinil, Epoprostenol, etc.); a peroxisome proliferator-activated receptor β/δ isoform (PPARδ) agonist (e.g., GW501516, also known as GW-501,516, GW1516, GSK-516, Endurobol, etc.); a cAMP inducer (e.g., forskolin, also known as coleonol, 8-bromo-cAMP, etc.); a phosphodiesterase inhibitors (e.g., sildenafil citrate (VIAGRA®), tadalafil (CIALIS®), vardenafil (LEVITRA®), etc.); an endothelin receptor antagonist (e.g., bosentan (TRACLEER®), ambrisentan (LETAIRIS®), macitentan (OPSUMIT®), etc.); a nitrous oxide modulating agent (e.g., nitrates, or soluble GMP cyclase inducers, such as for example riociguat (ADEMPAS)); a non-prostanoid IP receptor agonist (e.g., selexipag); and the like; or combinations thereof For purposes of the disclosure herein, an “effective amount” of composition for PGI2 delivery may be defined as an amount of composition for PGI2 delivery that produces a therapeutic response or desired effect (e.g., increase PGI2 levels in a body area) in some fraction of individuals to which it is administered. For purposes of the disclosure herein, a pharmaceutical composition generally refers to any composition that may be used on or in a body to prevent, deter, diagnose, alleviate, treat, and/or cure a disease in humans or animals.

In an embodiment, a method of treating an individual having a vascular disease or at risk of developing a vascular disease may comprise administering to the individual a pharmaceutical composition comprising an effective amount of the composition for PGI2 delivery, thereby ameliorating, deterring and/or preventing the vascular disease in said individual. In an embodiment, the composition for PGI2 delivery may be a pharmaceutical composition.

In an embodiment, the composition for PGI2 delivery may be administered to an individual, wherein the composition for PGI2 delivery comprises a plasmid comprising the cyclooxygenase-prostacyclin synthase fusion gene. In such embodiment, the plasmid may enter a host cell of the individual, and may induce PGI2 overexpression by the host cell, wherein the PGI2 may then be secreted into surrounding areas/tissues. In such embodiments, the host cell overexpresses PGI2.

In an embodiment, the composition for PGI2 delivery comprising the plasmid may be administered by injection, such as for example an intramuscular injection, an intra-arterial injection, etc.

In an embodiment, the composition for PGI2 delivery comprising the plasmid may be locally injected (e.g., intramuscular injection) in a body area. In an embodiment, the composition for PGI2 delivery comprising the plasmid may be locally injected (e.g., intramuscular injection) into a muscle tissue, such as for example a brachial skeletal muscle tissue, heart tissue, lung tissue, and the like, or combinations thereof In an embodiment, the composition for PGI2 delivery comprising the plasmid may be locally injected (e.g., intramuscular injection) in ischemic areas or tissues, such as for example ischemic heart tissue, ischemic kidney tissue, ischemic limb tissue, ischemic brain tissue (e.g., ischemic brain tissue associated with stroke or subarachnoid hemorrhage), and the like. In such embodiments, the plasmid may enter a host cell of the individual, and may induce PGI2 overexpression by the host cell, wherein the PGI2 may then be secreted into surrounding areas/tissues, thereby enhancing tissue vascularization and restoring blood flow into at least a portion of the ischemic tissue.

In an embodiment, a method of treating an individual having a vascular disease or at risk of developing a vascular disease may comprise administering to the individual an effective amount of a composition for PGI2 delivery, wherein the composition for PGI2 delivery comprises a plasmid comprising the cyclooxygenase-prostacyclin synthase fusion gene and the human muscle specific promoter and PBS, wherein the plasmid may be suspended in PBS, thereby ameliorating, deterring and/or preventing the vascular disease. In such embodiment, the vascular disease may comprise PAH and the composition for PGI2 delivery may be administered by local injection (e.g., intramuscular injection) into the ischemic tissue.

In another embodiment, a method of treating an individual having a vascular disease or at risk of developing a vascular disease may comprise administering to the individual an effective amount of a composition for PGI2 delivery, wherein the composition for PGI2 delivery comprises a plasmid comprising the cyclooxygenase-prostacyclin synthase fusion gene and the human muscle specific promoter and a liposome carrier, thereby ameliorating, deterring and/or preventing the vascular disease. In such embodiment, the vascular disease may comprise PAH and the composition for PGI2 delivery may be administered by intra-arterial injection.

In yet another embodiment, a method of treating an individual having pulmonary arterial hypertension or at risk of developing pulmonary arterial hypertension comprises administering to the individual an effective amount of a composition for PGI2 delivery; wherein the composition for PGI2 delivery comprises the plasmid comprising the cyclooxygenase-prostacyclin synthase fusion gene, wherein the plasmid comprises the human muscle specific promoter, and phosphate buffer saline; wherein the plasmid is present within the composition for PGI2 delivery in an amount of from about 0.4 mg/ml to about 500 mg/ml; and wherein the composition may be administered via an intramuscular injection into a brachial skeletal muscle; wherein the plasmid enters a host cell of the brachial skeletal muscle upon administering said composition to said individual; and wherein the host cell overexpresses prostacyclin.

In still yet another embodiment, a method of treating an individual having pulmonary arterial hypertension or at risk of developing pulmonary arterial hypertension comprises administering to the individual an effective amount of a composition for PGI2 delivery; wherein the composition for PGI2 delivery comprises the plasmid comprising the cyclooxygenase-prostacyclin synthase fusion gene, wherein the plasmid comprises the human muscle specific promoter, and a nanoparticle carrier; wherein the plasmid is present within the composition for PGI2 delivery in an amount of from about 0.4 mg/ml to about 500 mg/ml; and wherein the composition may be administered via an intra-arterial injection; wherein the plasmid enters a host cell of said individual; and wherein the host cell overexpresses prostacyclin.

In an embodiment, the method of treating an individual having a vascular disease or at risk of developing a vascular disease as disclosed herein advantageously displays improvements in one or more outcomes when compared to conventional treatment methods. In an embodiment, the method of administering the composition for PGI2 delivery may simplify delivery of a target gene (cyclooxygenase-prostacyclin synthase fusion gene) when compared to conventional methods of delivering such target gene. Conventionally, a target gene can generally be delivered by using viral vectors and/or by using cells as carriers.

In an embodiment, the method of treating an individual having a vascular disease or at risk of developing a vascular disease as disclosed herein may have several advantages over current standard PGI2 therapies. In an embodiment, the method of administering the composition for PGI2 delivery may advantageously induce PGI2 overexpression directly within ischemic tissue. In an embodiment, the method of administering the composition for PGI2 delivery may advantageously and consistently provide a high level of PGI2 to ischemic tissues.

In an embodiment, the method of administering the composition for PGI2 delivery may advantageously and effectively alleviate tissue ischemia and improve functional recovery. The composition for PGI2 delivery as disclosed herein may advantageously provide a way to specifically increase the biosynthesis of the vascular protector PGI2 in ischemic tissue, and as such is believed to be an important development in pharmacology.

In an embodiment, the composition for PGI2 delivery may advantageously allow for direct in vivo synthesis of the potent vascular protector, PGI2, from AA with a high efficiency, which may be used to prevent and rescue patients from vascular diseases (e.g., PAH, PAD, peripheral vascular disease, COPD, ischemia, limb ischemia, CLI, Reynaud's syndrome, ischemic stroke, myocardial infarction, systemic hypertension, etc.) through specifically increasing PGI2 production in target areas, such as for example in ischemic tissue. Additional advantages of the composition for PGI2 delivery and treatment methods of using same may be apparent to one of skill in the art viewing this disclosure.

### EXAMPLES

The embodiments having been generally described, the following examples are given as particular embodiments of the disclosure and to demonstrate the practice and advantages thereof It is understood that the examples are given by way of illustration and are not intended to limit the specification or the claims in any manner.

### Example 1

The outcomes of plasmid injections in monocrotaline (MCT) induced rat PAH model were investigated. More specifically, the effect of injections with a plasmid containing a cyclooxygenase-prostacyclin synthase fusion gene were monitored in a MCT induced rat PAH model.

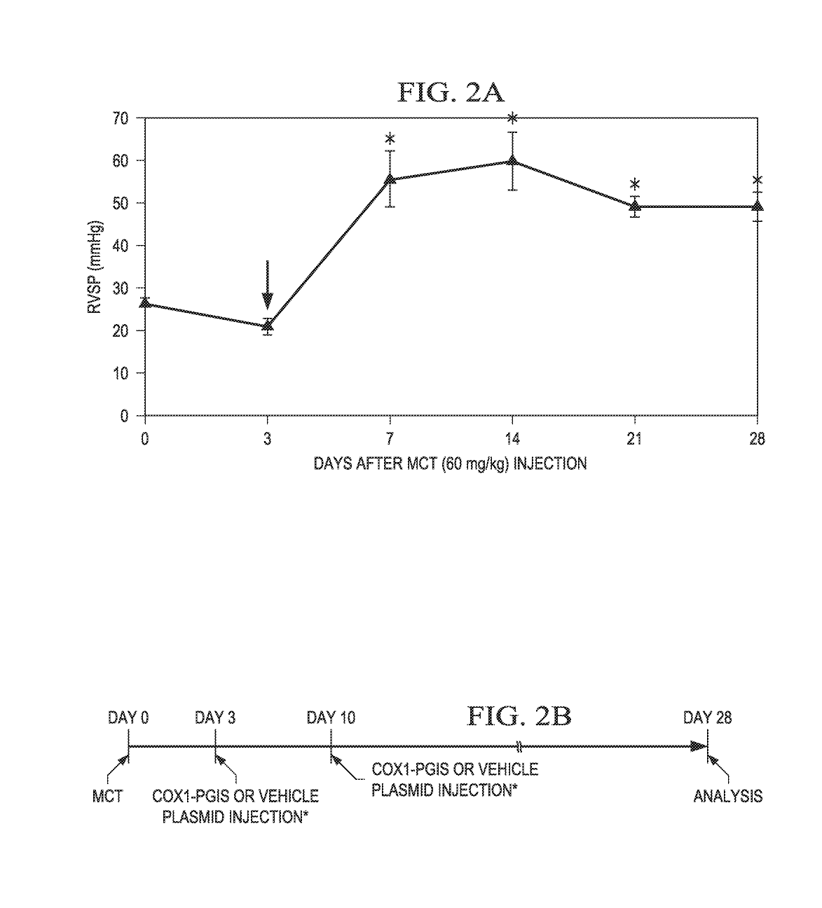

MCT (60 mg/kg; Sigma-Aldrich Corporation, St. Louis, Mo.) was intraperitoneally injected into 6-week-old male Fisher 344 rats. The right ventricular systolic pressure (RVSP) was monitored for 28 days following the MCT injection, and the results are displayed in FIG. 2A. FIG. 2A displays a graph of RVSP over a time period of 28 days in rats injected with MCT. A week after the MCT injection, the RVSP almost doubles, and it remains at an elevated value until the end of the monitored period.

At 3 days and 10 days after MCT injection, the rats were injected with either the plasmid containing the cyclooxygenase-prostacyclin synthase fusion gene or the vehicle plasmid, as seen in FIG. 2B. FIG. 2B displays a schematic of an MCT-induced rat PAH model during a study with plasmid containing the fusion gene. The plasmid was administered to the rats by brachial intramuscular injection at a concentration of 2 mg/mL of either plasmid containing the fusion gene or vehicle plasmid. The volume injected was 250 □L and it contained 500 □g of either plasmid containing the fusion gene or vehicle plasmid. The carrier fluid was PBS. Analysis was performed at various time points across a 28 day period, such as for example 1 week, 2 weeks, 3 weeks, and 4 weeks. A PGI2 synthetic analogue, Treprostinil, was used as a comparison. Treprostinil was administered daily to the rats at 0.5 mg/kg body weight.

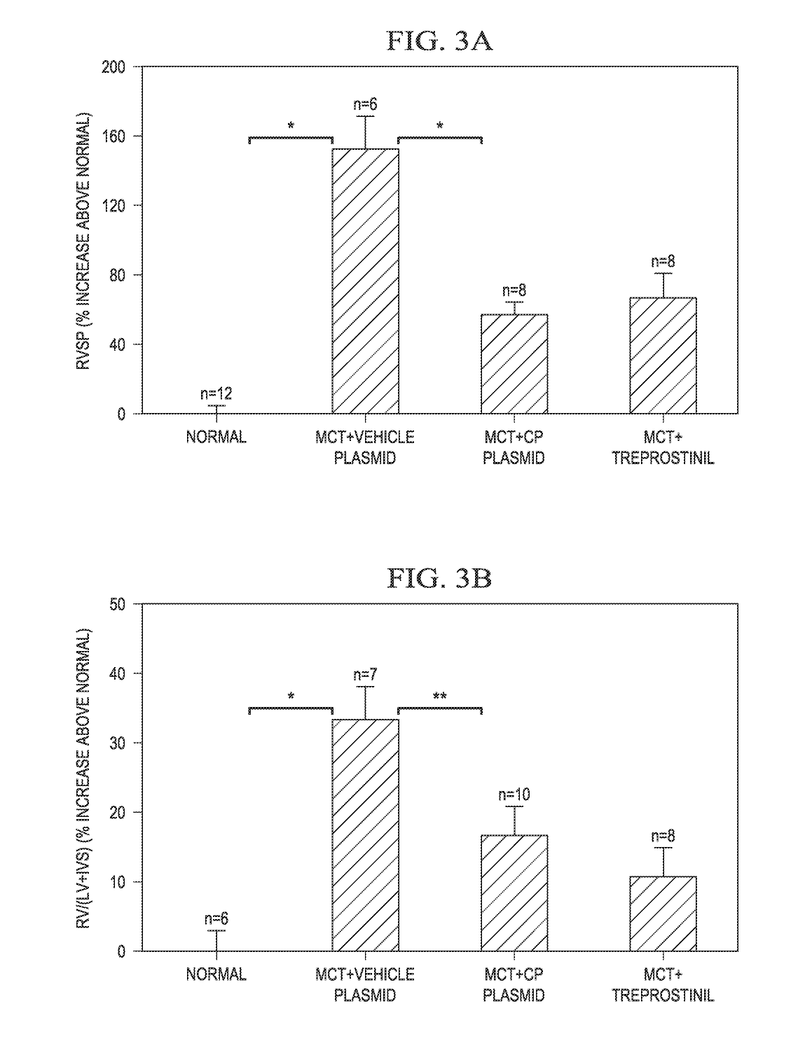

The intramuscular injection of plasmid containing the fusion gene at 3 days and 10 days after MCT injection (FIG. 2B) prevented the MCT induced RVSP increase, as seen in FIG. 3A. FIG. 3A displays a graph of RVSP % increase in various treatment groups of a MCT induced rat PAH model. “CP” denotes the COX-1-linker-PGIS fusion gene. The % increase above normal was calculated as (mean of treated group mean of normal group)/mean of normal group. The group that was treated with the vehicle plasmid exhibited the greatest increase in RVSP. The groups that were treated with either CP plasmid or Treprostinil displayed a much lower (less than half) increase in RVSP when compared to the group that was treated with the vehicle plasmid (*P<0.01).

Right ventricle (RV) hypertrophy was also monitored in all treatment groups, and the results are shown in FIG. 3B. FIG. 3B displays a graph of RV hypertrophy % increase in various treatment groups of a MCT induced rat PAH model, wherein RV hypertrophy is calculated as RV/(LV+IVS), wherein “LV” denotes left ventricle, and “IVS” denotes interventricular septum. The % increase above normal was calculated as (mean of treated group mean of normal group)/mean of normal group. Similarly to RVSP, the group that was treated with the vehicle plasmid exhibited the greatest increase in RV hypertrophy. The groups that were treated with either CP plasmid or Treprostinil displayed a much lower (less than half) increase in RV hypertrophy when compared to the group that was treated with the vehicle plasmid (*P<0.01; **P<0.05).

Clinical observations indicated that RVSP does not strongly correlate with survival in PAH patients, whereas RV mass and function are strong predictors of survival. The RVSP and RV correlations with survival in PAH patients are described in more detail in Champion, H. C.; Michelakis, E. D.; Hassoun, P. M. “Comprehensive invasive and noninvasive approach to the right ventricle-pulmonary circulation unit: state of the art and clinical and research implications.” Circulation 2009; vol. 120; pp 992-1007; and Voelkel, N. F.; Quaife, R. A.; Leinwand, L.A.; Barst, R. I.; McGoon, M. D.; Meldrum, D. R.; Dupuis, J.; Long, C S.; Rubin, L. J.; Smart, F. W.; Suzuki, Y. J.; Gladwin, M.; Denholm, E. M.; Gail, D. B.; “Report of a National Heart, Lung, and Blood Institute Working Group on Cellular and Molecular Mechanisms of Right Heart Failure.” Circulation 2006; vol. 114; pp 1883-1891; each of which is incorporated by reference herein in its entirety.

Since it was found that engineered ELPCs carrying the COX1-PGIS expressing plasmid could prevent and reverse RV hypertrophy and increase the survival of PAH rats, and FIG. 3B displays an in improvement of RV hypertrophy with the COX1-PGIS plasmid, treatment with naked COX1-PGIS plasmid may also provide survival benefit to PAH similar to that of the engineered ELPCs. The study regarding engineered ELPCs carrying the COX1-PGIS expressing plasmid that could prevent and reverse RV hypertrophy and increase the survival of PAH rats is described in more detail in Zhou, L.; Chen, Z.; Vanderslice, P.; So, S. P.; Ruan, K. H.; Willerson, J. T.; Dixon, R. A. “Endothelial-like progenitor cells engineered to produce prostacyclin rescue monocrotaline-induced pulmonary arterial hypertension and provide right ventricle benefits.” Circulation 2013, vol. 128(9); pp 982-994, which is incorporated by reference herein in its entirety.

### Example 2

The outcomes of plasmid injections in MCT induced rat PAH model were investigated. More specifically, the effect of injections with a plasmid containing a cyclooxygenase-prostacyclin synthase fusion gene were investigated by comparison to injections of a vehicle plasmid in a MCT induced rat PAH model. The MCT induced rat PAH model and the plasmid injections were designed and conducted as described in Example 1.

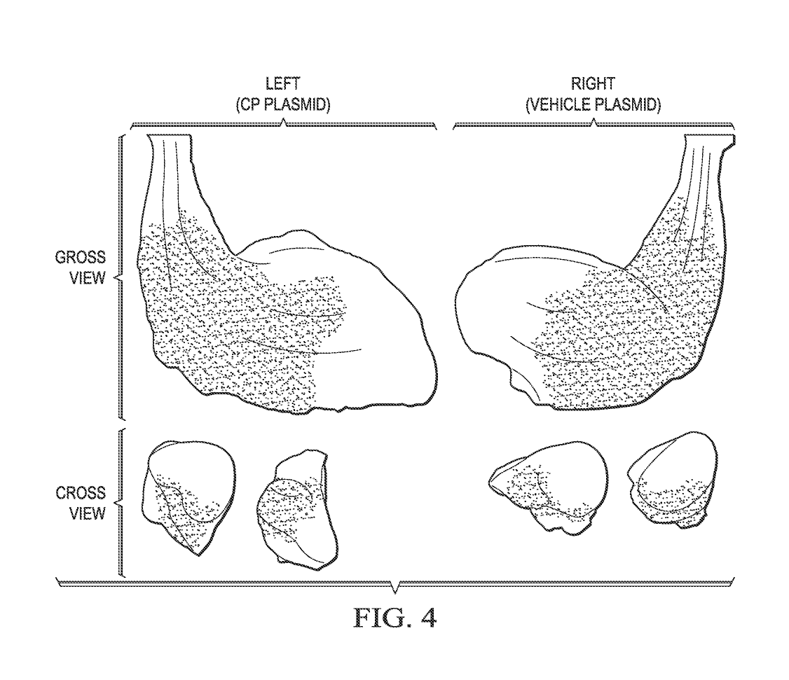

The brachial muscle that was injected with either plasmid containing the fusion gene or vehicle plasmid was analyzed two weeks after the plasmid injection, as seen in FIG. 4. 0.4% Trypan blue was used as an injection indicator. FIG. 4 displays images of brachial muscles of various treatment groups of a MCT induced rat PAH model, both as a gross view and as a cross view. FIG. 4 displays areas that were stained by Trypan blue.

### Example 3

The outcomes of plasmid injections in MCT induced rat PAH model were investigated. More specifically, the effect of injections with a plasmid containing a cyclooxygenase-prostacyclin synthase fusion gene were investigated by comparison to injections of a vehicle plasmid in a MCT induced rat PAH model. The MCT induced rat PAH model and the plasmid injections were designed and conducted as described in Example 1.

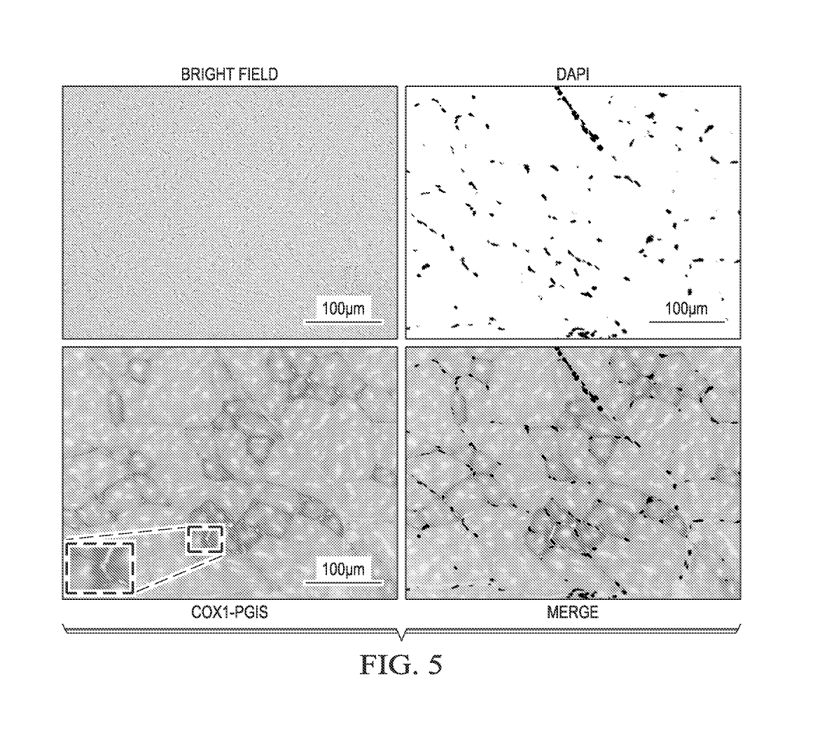

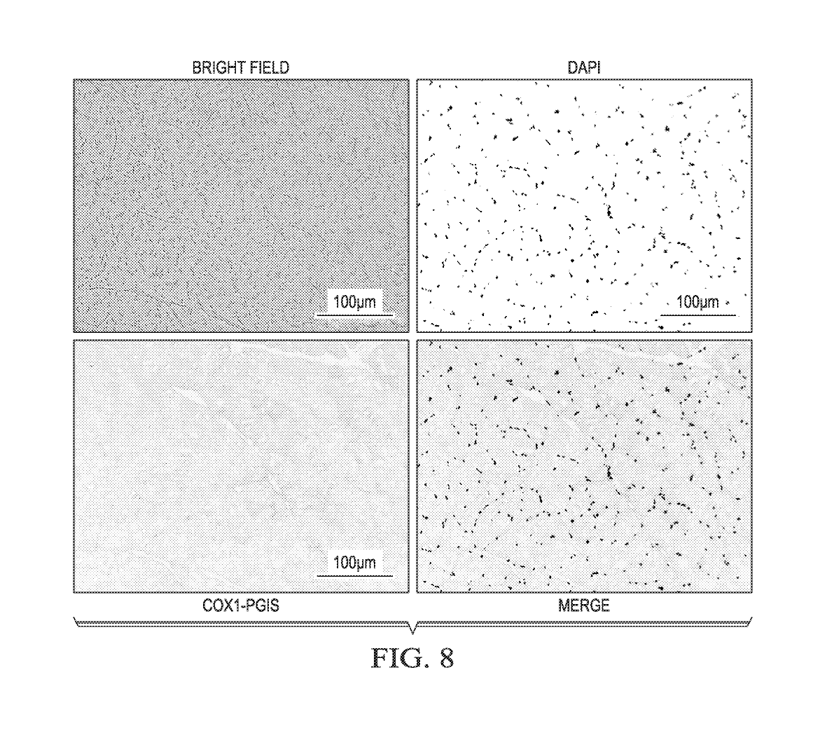

The muscle tissue that was injected with plasmid (either plasmid containing the fusion gene or vehicle plasmid) was analyzed by microscopy, and the results are displayed in FIGS. 5, 6, 7, 8, and 9; wherein the top left image is a bright field image; the top right image is a fluorescence microscopy image for DAPI staining, the bottom left image is a fluorescence microscopy image for specific antibody staining (e.g., FIGS. 5, 6, 7, and 9 display fluorescence imaging of an antibody that specifically recognizes human PGIS; and FIG. 8 displays fluorescence imaging of isotype IgG as a control); and the bottom right image is a merged image of the DAPI staining and the specific antibody staining. DAPI staining shows nuclei.

FIG. 5 displays microscope images of a cross section of a MCT-rat brachial skeletal muscle, two weeks after injection with the plasmid containing the fusion gene (e.g., naked plasmid, naked COX-1-linker-PGIS plasmid). The merged image of FIG. 5 indicates that the nuclei (stained with DAPI) and the fusion gene (stained with the antibody that specifically recognizes human PGIS) are part of the same cellular structures, indicating plasmid uptake by the cells and increased levels of the fusion gene (PGIS) in these cells.

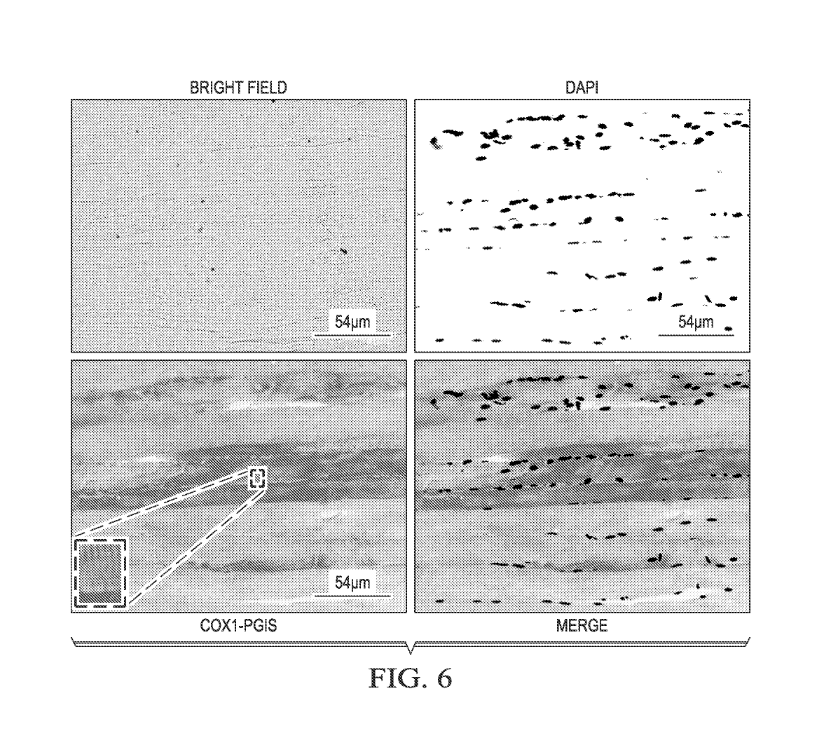

FIG. 6 displays microscope images of a longitudinal section of a MCT-rat brachial skeletal muscle, two weeks after injection with the plasmid containing the fusion gene (e.g., naked plasmid, naked COX-1-linker-PGIS plasmid). The merged image of FIG. 6 indicates that the nuclei (stained with DAPI) and the fusion gene (stained with the antibody that specifically recognizes human PGIS) are part of the same cellular structures, indicating plasmid uptake by the cells and increased levels of the fusion gene (PGIS) in these cells.

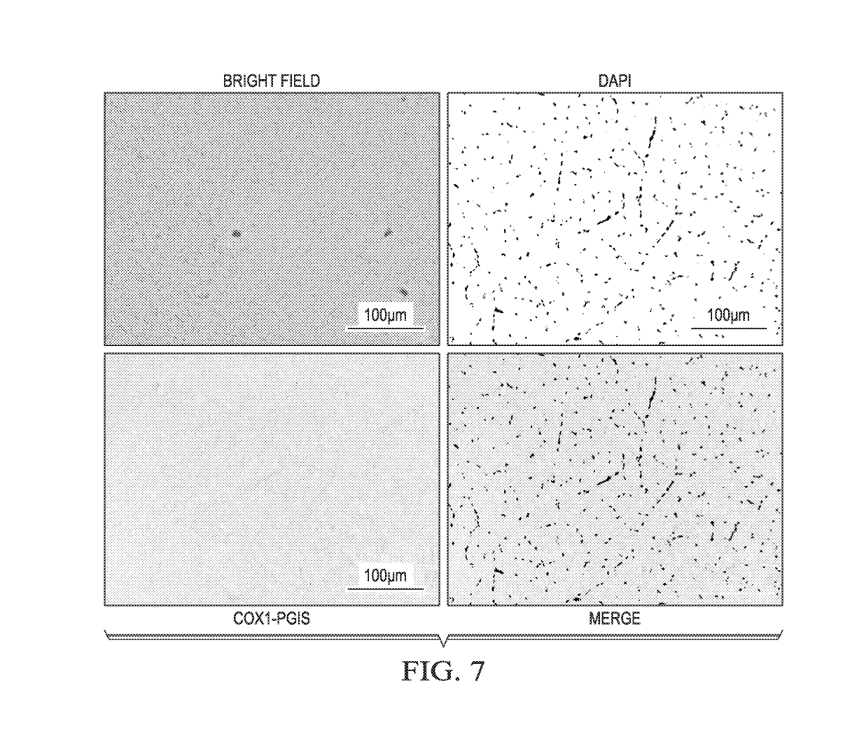

FIG. 7 displays microscope images of a section of a MCT-rat brachial skeletal muscle, two weeks after injection with vehicle plasmid (e.g., naked vehicle control plasmid). FIG. 7 displays no visible staining for the fusion gene (stained with the antibody that specifically recognizes human PGIS), indicating that the antibody is really specific for human PGIS and does not yield false positive staining.

FIG. 8 displays microscope images of a section of a MCT-rat brachial skeletal muscle, two weeks after injection with plasmid, isotype IgG control, wherein the plasmid contains the fusion gene. FIG. 8 displays no visible staining for the isotype IgG.

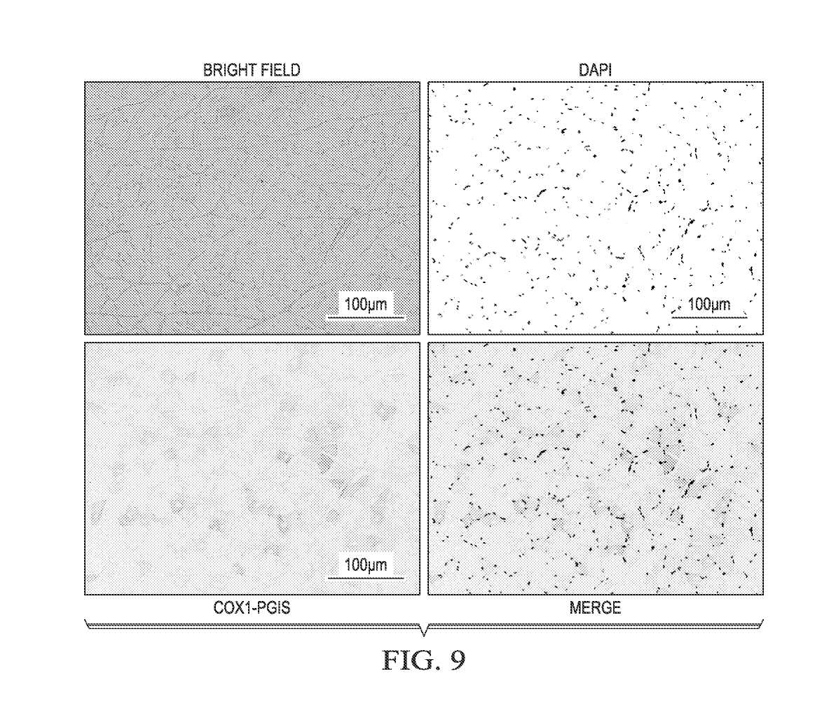

FIG. 9 displays microscope images of a section of a MCT-rat brachial skeletal muscle, three weeks after injection with plasmid, wherein the plasmid contains a fusion gene. FIG. 9 displays a weak staining for the fusion gene (stained with the antibody that specifically recognizes human PGIS), even after three weeks from the injection time, indicating that the plasmid injection causes an increase in PGIS in the tissue, wherein increased PGIS levels of are maintained over time.

### ADDITIONAL DISCLOSURE

The following are nonlimiting, specific embodiments in accordance with the present disclosure:

A first embodiment, which is an effective amount of a composition comprising (i) a plasmid having a cyclooxygenase-prostacyclin synthase fusion gene, and (ii) a carrier fluid for use in treating an individual having a vascular disease or at risk of developing a vascular disease.

A second embodiment, which is the composition of the first embodiment wherein the vascular disease comprises: pulmonary arterial hypertension, peripheral arterial disease, peripheral vascular disease, chronic obstructive pulmonary disease, ischemia, limb ischemia, critical limb ischemia, Reynaud's syndrome, ischemic stroke, myocardial infarction, systemic hypertension, stroke, subarachnoid hemorrhage, or combinations thereof.

A third embodiment, which is the composition of any of the first or the second embodiments wherein the plasmid comprises a DNA sequence encoding for a triple catalytic enzyme.

A fourth embodiment, which is the composition of the third embodiment wherein the triple catalytic enzyme is characterized by a formula COX-linker-PGIS, wherein COX comprises a cyclooxygenase (COX) amino acid sequence; PGIS is prostacyclin synthase; and the linker comprises from about 10 to about 22 amino acid residues of a transmembrane sequence; wherein the linker is disposed between the COX and PGIS, and wherein the linker directly connects the COX to the PGIS.

A fifth embodiment, which is the composition of the fourth embodiment wherein the triple catalytic enzyme is characterized by a formula COX-1-10aa-PGIS; wherein COX-1 is cyclooxygenase isoform-1; the linker comprises a 10 amino acid (10aa) transmembrane sequence; and PGIS is prostacyclin synthase.

A sixth embodiment, which is the composition of any of the first through the fifth embodiments wherein the plasmid comprises a constitutively active mammalian promoter.

A seventh embodiment, which is the composition of any of the first through the sixth embodiments wherein the plasmid comprises an inducible promoter.

An eighth embodiment, which is the composition of any of the first through the seventh embodiments wherein the plasmid comprises a human muscle specific promoter, human muscle creatinine kinase promoter, human □-skeletal actin promoter, human desmin promoter, human troponin I promoter, human cytomegalovirus promoter, antibiotic-inducible promoter, tetracycline-inducible promoter, doxycycline-inducible promoter, bacitracin-inducible promoter, vancomycin-inducible promoter, and isopropyl beta-D-thiogalactopyranoside (IPTG)-inducible promoter.

A ninth embodiment, which is the composition of any of the first through the eighth embodiments administered via an intramuscular injection.

A tenth embodiment, which is the composition of any of the first through the eighth embodiments wherein the composition is injected into an ischemic tissue.

An eleventh embodiment, which is the composition of any of the first through the tenth embodiments wherein the vascular disease comprises pulmonary arterial hypertension, and wherein the composition is injected into a brachial skeletal muscle.

A twelfth embodiment, which is the composition of any of the first through the eleventh embodiments wherein the plasmid enters a host cell upon administering said composition to said individual, and wherein said host cell overexpresses prostacyclin (PGI2).

A thirteenth embodiment, which is the composition of any of the first through the twelfth embodiments wherein the carrier fluid comprises phosphate buffer saline.

A fourteenth embodiment, which is the composition of any of the first through the thirteenth embodiments wherein the plasmid is present within the composition in an amount of from about 0.4 mg/ml to about 500 mg/ml.

A fifteenth embodiment, which is the composition of any of the first through the fourteenth embodiments excluding a viral vector.

A sixteenth embodiment, which is a composition comprising a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof, for use in treating an individual having a vascular disease or at risk of developing a vascular disease.

A seventeenth embodiment, which is the composition of the sixteenth embodiment administered via an intramuscular injection into an ischemic tissue.

An eighteenth embodiment, which is the composition of any of the sixteenth or the seventeenth embodiments wherein the triple catalytic enzyme is characterized by a formula COX-1-10aa-PGIS; wherein COX-1 is cyclooxygenase isoform-1; the linker comprises a 10 amino acid (10aa) transmembrane sequence; and PGIS is prostacyclin synthase.

A nineteenth embodiment, which is a composition comprising a plasmid comprising a cyclooxygenase-prostacyclin synthase fusion gene, wherein the plasmid comprises a human muscle specific promoter, and phosphate buffer saline, for use in treating an individual having pulmonary arterial hypertension or at risk of developing pulmonary arterial hypertension, wherein the plasmid is present within the composition in an amount of from about 0.4 mg/ml to about 500 mg/ml; and wherein the composition is administered via an intramuscular injection into a brachial skeletal muscle.

A twentieth embodiment, which is a composition for prostacyclin (PGI2) delivery, wherein the composition for PGI2 delivery comprises: a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof.

A twenty-first embodiment, which is the composition of the twentieth embodiment, wherein the plasmid is present within the composition in an amount of from about 0.4 mg/ml to about 500 mg/ml.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Detailed Description of the Embodiments is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. An effective amount of a composition comprising (i) a plasmid having a cyclooxygenase-prostacyclin synthase fusion gene, and (ii) a carrier fluid for use in treating an individual having a vascular disease or at risk of developing a vascular disease.

2. The composition of claim 1 wherein the vascular disease comprises: pulmonary arterial hypertension, peripheral arterial disease, peripheral vascular disease, chronic obstructive pulmonary disease, ischemia, limb ischemia, critical limb ischemia, Reynaud's syndrome, ischemic stroke, myocardial infarction, systemic hypertension, stroke, subarachnoid hemorrhage, or combinations thereof.

3. The composition of claim 1 wherein the plasmid comprises a DNA sequence encoding for a triple catalytic enzyme.

4. The composition of claim 3 wherein the triple catalytic enzyme is characterized by a formula COX-linker-PGIS, wherein COX comprises a cyclooxygenase (COX) amino acid sequence; PGIS is prostacyclin synthase; and the linker comprises from about 10 to about 22 amino acid residues of a transmembrane sequence; wherein the linker is disposed between the COX and PGIS, and wherein the linker directly connects the COX to the PGIS.

5. The composition of claim 4 wherein the triple catalytic enzyme is characterized by a formula COX-1-10aa-PGIS; wherein COX-1 is cyclooxygenase isoform-1; the linker comprises a 10 amino acid (10aa) transmembrane sequence; and PGIS is prostacyclin synthase.

6. The composition of claim 1 wherein the plasmid comprises a constitutively active mammalian promoter.

7. The composition of claim 1 wherein the plasmid comprises an inducible promoter.

8. The composition of claim 1 wherein the plasmid comprises a human muscle specific promoter, human muscle creatinine kinase promoter, human a-skeletal actin promoter, human desmin promoter, human troponin I promoter, human cytomegalovirus promoter, antibiotic-inducible promoter, tetracycline-inducible promoter, doxycycline-inducible promoter, bacitracin-inducible promoter, vancomycin-inducible promoter, and isopropyl beta-D-thiogalactopyranoside (IPTG)-inducible promoter.

9. The composition of claim 1 administered via an intramuscular injection.

10. The composition of claim 1 wherein the composition is injected into an ischemic tissue.

11. The composition of claim 1 wherein the vascular disease comprises pulmonary arterial hypertension, and wherein the composition is injected into a brachial skeletal muscle.

12. The composition of claim 1 wherein the plasmid enters a host cell upon administering said composition to said individual, and wherein said host cell overexpresses prostacyclin (PGI2).

13. The composition of claim 1 wherein the carrier fluid comprises phosphate buffer saline.

14. The composition of claim 1 wherein the plasmid is present within the composition in an amount of from about 0.4 mg/ml to about 500 mg/ml.

15. The composition of claim 1 excluding a viral vector.

16. A composition comprising a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof, for use in treating an individual having a vascular disease or at risk of developing a vascular disease.

17. The composition of claim 16 administered via an intramuscular injection into an ischemic tissue.

18. The composition of claim 16 wherein the triple catalytic enzyme is characterized by a formula COX-1-10aa-PGIS; wherein COX-1 is cyclooxygenase isoform-1; the linker comprises a 10 amino acid (10aa) transmembrane sequence; and PGIS is prostacyclin synthase.

19. A composition comprising a plasmid comprising a cyclooxygenase-prostacyclin synthase fusion gene, wherein the plasmid comprises a human muscle specific promoter, and phosphate buffer saline, for use in treating an individual having pulmonary arterial hypertension or at risk of developing pulmonary arterial hypertension, wherein the plasmid is present within the composition in an amount of from about 0.4 mg/ml to about 500 mg/ml; and wherein the composition is administered via an intramuscular injection into a brachial skeletal muscle.

20. A composition for prostacyclin (PGI2) delivery, wherein the composition for PGI2 delivery comprises: a carrier fluid; and a DNA sequence encoding for a triple catalytic enzyme, a cDNA sequence encoding for a triple catalytic enzyme, a plasmid comprising a DNA sequence encoding for a triple catalytic enzyme, a fusion gene encoding for a triple catalytic enzyme, a cyclooxygenase-prostacyclin synthase fusion gene, or combinations thereof.

21. The composition of claim 20, wherein the plasmid is present within the composition in an amount of from about 0.4 mg/ml to about 500 mg/ml.
